(19) United States
(12) Patent Application Publication
(10) Pub. No.: US 2025/0022589 A1
(43) Pub. Date: Jan. 16, 2025

(54) SYSTEM AND METHOD FOR FACILIATING HEALTHCARE WATER CONTROL POINT MAINTENANCE AND COMPLIANCE

(71) Applicant: Water Saver Solutions, Inc., Santa Fe Springs, CA (US)

(72) Inventors: Gregory Paul, Los Angeles, CA (US); Dustin Gordon, Zephyrhills, FL (US)

(21) Appl. No.: 18/772,251

(22) Filed: Jul. 14, 2024

Related U.S. Application Data
(60) Provisional application No. 63/526,813, filed on Jul. 14, 2023.

(51) Int. Cl. G16H 40/20; H04L 9/40

(52) U.S. Cl. CPC G16H 40/20; H04L 63/083

(57) ABSTRACT

A system and method for facilitating healthcare water control point maintenance and compliance includes a server in network communication with user devices of technician users and supervisory users. A server database stores room data for each of a plurality of rooms, including a room identifier, group identifiers of location groups with which the room is associated, types of water control points within the room, prescribed service interval for each of the types of water control points, quantity of each type of water control point within the room, service completion status, and date of last service completion. Technician users are able to review rooms in which water control point service is required, and input information about the status of the control points they service. The supervisory users can view status data concerning each water control point based on various selectable location levels, including room, floor, building, campus and company levels.

### RELATED APPLICATIONS

This application claims the benefit of U.S. Provisional Application No. 63/526,813 filed Jul. 14, 2023, the contents of which are incorporated by this reference their entirety for all purposes as if fully set forth herein.

### TECHNICAL FIELD

The disclosure herein relates generally to computer-based systems and methods to assist in servicing a large number of water control points.

### BACKGROUND

In healthcare, infection risk management is critical. A component of such risk management includes performing preventive maintenance of water control points like replacing filters or laminar flow devices at water faucets, showers and ice machines within healthcare facilities on a regular basis. Moreover, owners and operators of healthcare facilities would like to be able to verify and provide evidence that their facilities are in compliance with the relevant laws and regulations relating to water control point care.

What is needed is a system for efficiently facilitating water control point maintenance and associated compliance verification, particularly within the healthcare environment, for use across numerous healthcare facilities, campuses and geographical locations.

### SUMMARY

One or more deficiencies of the prior art are solved by way of embodiments of the system and method for facilitating water control point maintenance and compliance in accordance with the present disclosure.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 depicts schematic view of an exemplary system for facilitating healthcare water control point maintenance and compliance;

FIG. 2 is a diagrammatic illustration of an example room location group hierarchy, wherein each company-level group includes respective campus-level groups, each campus-level group includes respective building-level groups, each building-level group includes respective floor-level groups, and each floor-level group includes respective rooms with one or more water control points therein;

FIG. 3 is a diagrammatic illustration of two different example room datasets, each room dataset including room data corresponding to a respective room;

FIG. 4 is a diagrammatic illustration of one example user account dataset;

FIG. 5 is an example of a message transmitted to a user device by way of an electronic address, the message including an account registration link;

FIG. 6 is a diagrammatic process flow chart illustrating one example of a set of server operations;

FIG. 7 is a diagrammatic process flow chart illustrating one example of a set of user device operations for a technician user device;

FIG. 8 is a diagrammatic example of a login page of the application interface that operates on a user device in accordance with the present disclosure;

FIG. 9 is a diagrammatic example of a control point status display page displaying control point status information for various company-level groups;

FIG. 10 is a diagrammatic example of a control point status display page similar to that of FIG. 9, but wherein the user has selected one of the hospitals on the display screen in order to bring up more detailed water control point status information pertaining to that hospital;

FIG. 11 is a diagrammatic example of a control point status display page similar to that of FIG. 9, but wherein a technician user with corresponding privileges has selected an option to add a company to be serviced by way of the system for facilitating healthcare water control point maintenance and compliance;

FIG. 12 is a diagrammatic example of a control point status display page displaying control point status information for the various campus-level groups within a specific selected company-level group;

FIG. 13 is a diagrammatic example of a control point status display page displaying control point status information for the various building-level groups within a specific selected campus-level group;

FIG. 14 is a diagrammatic example of a control point status display page displaying control point status information for the various building-level groups within a specific selected campus-level group, but wherein a drop-down menu has been selected;

FIG. 15 is a diagrammatic example of a room add page accessible by only a technician user;

FIG. 16 is a diagrammatic example of a room edit page with a room edit interface which may only be accessible by a technician user;

FIG. 17 is a diagrammatic example of a first portion of a room edit interface page which has been expanded to focus exclusively on a specific room selected by the technician user;

FIG. 18 is a diagrammatic example of a second portion of a room edit interface page which has been expanded to focus exclusively on a specific room selected by the technician user;

FIG. 19 is a diagrammatic example of a third portion of a room edit interface page which has been expanded to focus exclusively on a specific room selected by the technician user, this port allowing the technician user to enter observations which will be pushed as alerts to the user device of a supervisory user;

FIG. 20 is a diagrammatic example of a manage sinks page which may be accessible on the user device of a technician user;

FIG. 21 is a diagrammatic example of an add departments page which may be accessible on the user device of a technician user;

FIG. 22 is a diagrammatic example of a departments list page, which is control point status display page displaying control point status information for the various department-level groups within a specific selected floor-level group;

FIG. 23 is a diagrammatic example of an alerts page which may be accessible on the user device of a supervisory user, the alerts on this page originating from observations entered by the technician user for the respective rooms.

FIG. 24 is a diagrammatic example of an alerts resolution page (alerts resolve action page) which may be accessible on the user device of a supervisory user;

FIG. 25 is a diagrammatic example of an alerts resolution page (alerts resolve action page) with keyboard for the supervisory user to enter relevant alert resolution information;

FIG. 26 is a diagrammatic example of an alerts resolved notification page which the supervisory user may see once they have submitted the alert resolution information;

FIG. 27 is a diagrammatic example of an alerts page similar to that of FIG. 23, but wherein resolved alerts are indicated;

FIG. 28 is a diagrammatic example of an offline campuses page with user-selectable offline mode options for specific campuses;

FIG. 29 is a diagrammatic example of a first page of report publication interface provided by way of the administrator portal;

FIG. 30 is a diagrammatic example of a subsequent page of the report publication interface provided by way of the administrator portal;

FIG. 31 is one example first page of a service report published in accordance with the present disclosure;

FIG. 32 is one example second page of a service report published in accordance with the present disclosure;

FIG. 33 is one example third page of a service report published in accordance with the present disclosure;

FIG. 34 is one example fourth page of a service report published in accordance with the present disclosure;

FIG. 35 is one example sixth page of a service report published in accordance with the present disclosure;

FIG. 36 is one example seventh page of a service report published in accordance with the present disclosure;

FIG. 37 is one example of a conventional sink laminar flow device installed in a sink faucet;

FIG. 38 is one example of a conventional removable showerhead installed in a shower;

FIG. 39 is one example of a conventional sink filter installed in a sink faucet;

FIG. 40 is one example of a conventional ice machine filter; and

FIG. 41 is one example of a conventional replaceable showerhead assembly.

### DETAILED DESCRIPTION OF THE PREFERRED EMBODIMENTS

Referring now to the drawings, like reference numerals designate identical or corresponding features throughout the several views.

With reference to the several drawings, embodiments of a system for facilitating healthcare water control point maintenance and compliance are shown generally 100.

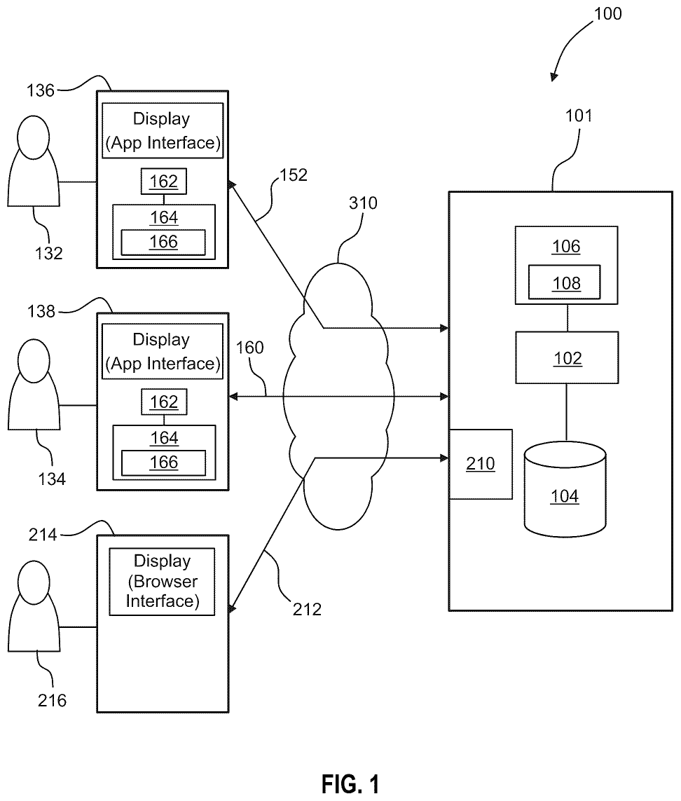

Referring to FIG. 1, preferred implementations of a system for facilitating healthcare water control point maintenance and compliance 100 may comprise a server processor 102, a server database 104 and a server memory 106. The server database 104 may be operably coupled with the server processor 102.

The server memory 106, which is not a transitory propagating signal, may be configured to be operably connected to the server processor 102 and include server processor executable instructions 108. The server processor executable instructions 108, when executed by the server processor 102, cause the server processor 102 to perform server operations 110.

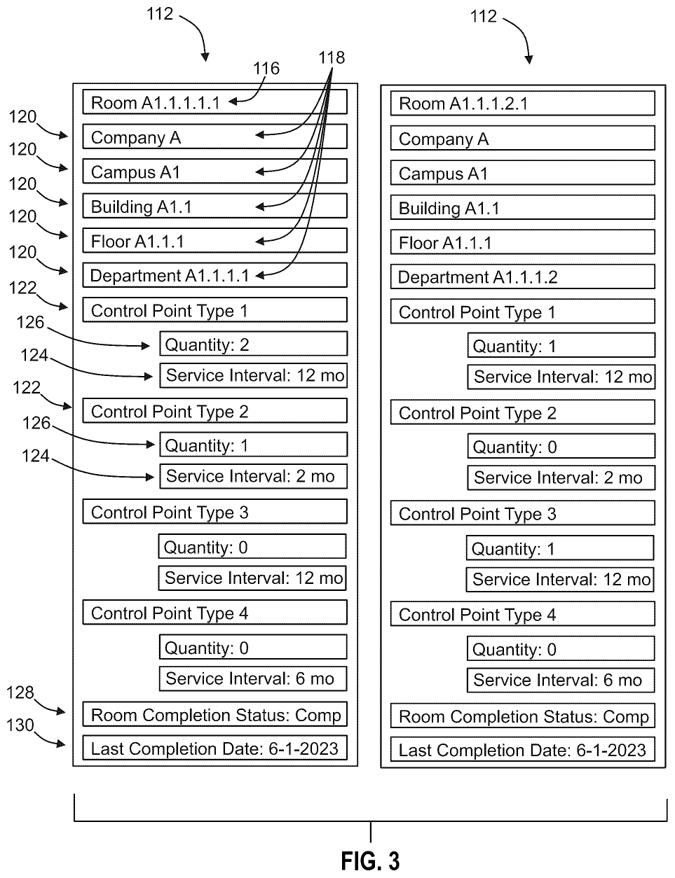

Referring to FIG. 3, the server operations 110 may comprise storing, to the server database 104, room data 112 for each of a plurality of rooms 114. The room data 112 may include, for example, a room identifier 116, group identifiers 118 of location groups 120 with which the room 114 is associated, types of water control points 122 within the room 114, prescribed service interval 124 for each of the types of water control points 122, quantity 126 of each type of water control point 122 within the room 114, service completion status 128, and date of last service completion 130.

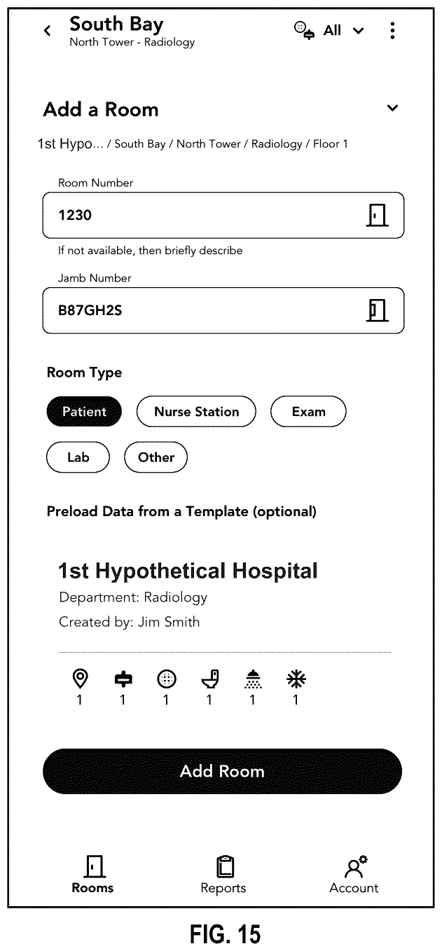

The room identifier may be, for example, a room number, a jamb number or a room location description. Referring to FIG. 15, in preferred implementations of the system 100, if no room number or jamb number is available, the technician user serving the room will be required to enter a room location description such as a very brief narrative describing the specific location in which the water control points reside. Moreover, in certain circumstances, a “room” for the purpose of the present system 100 may not always be in the form of an enclosable space. Rather, occasionally the room 114 may be a portion of a hallway, a location by an elevator, etc.

In preferred implementations of the system 100, the server operations 110 may comprise recording, for each of a plurality of users (e.g., 132, 134) having a user device (e.g., 136, 138), a user account data set 140. The user account data set 140 may include user account setup data 142 and login credentials 144 of the user (e.g., 132, 134). The user account setup data 142 may include a user type 146, a user identity 148 and location permissions 150. The user type 146 may be either technician user or supervisory user. The location permissions 150 may correspond to one or more of the rooms 114.

In preferred implementations of the system 100, for each of one or more technician users 132, the server operations 110 may comprise maintaining an authenticated connection 152 between the server database 104 and the user device 136 of the technician user 132. The authenticated connection 152 is preferably based upon the login credentials 144 of the technician user 132. Notably, the user devices (136, 138) of the technician users and the supervisory users would be operating a software application specifically configured to interface with the server 101 of the system 100 by way of the respective authenticated connections (152, 160). In preferred implementations, the software application would be the same for the technician user device 136 and the supervisory user device 138.

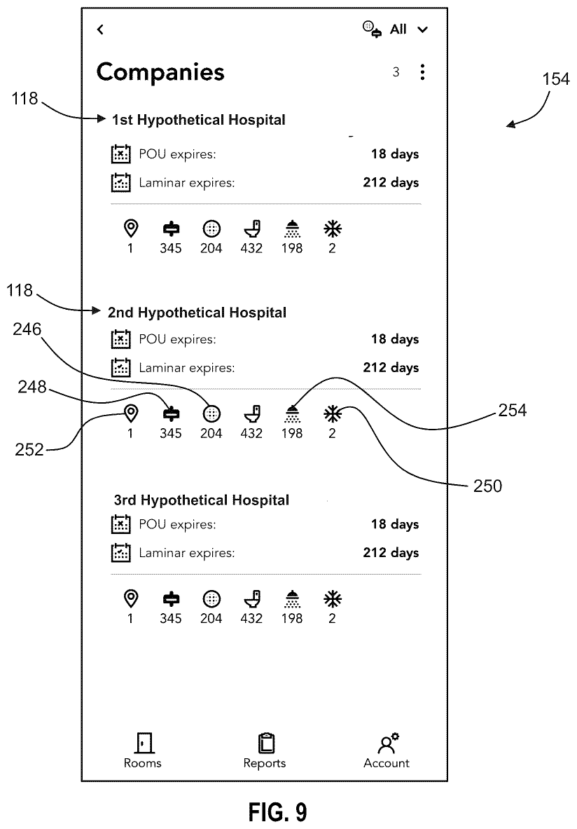

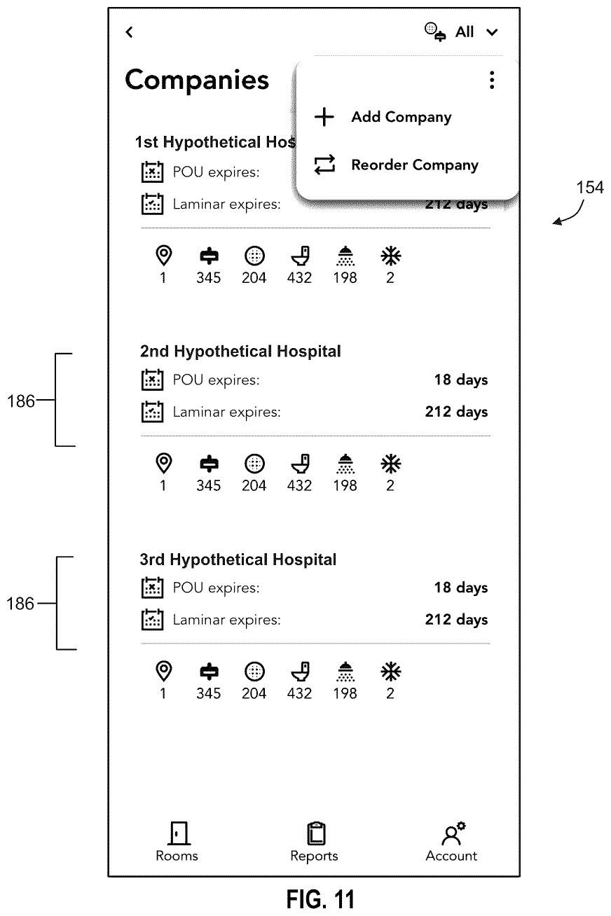

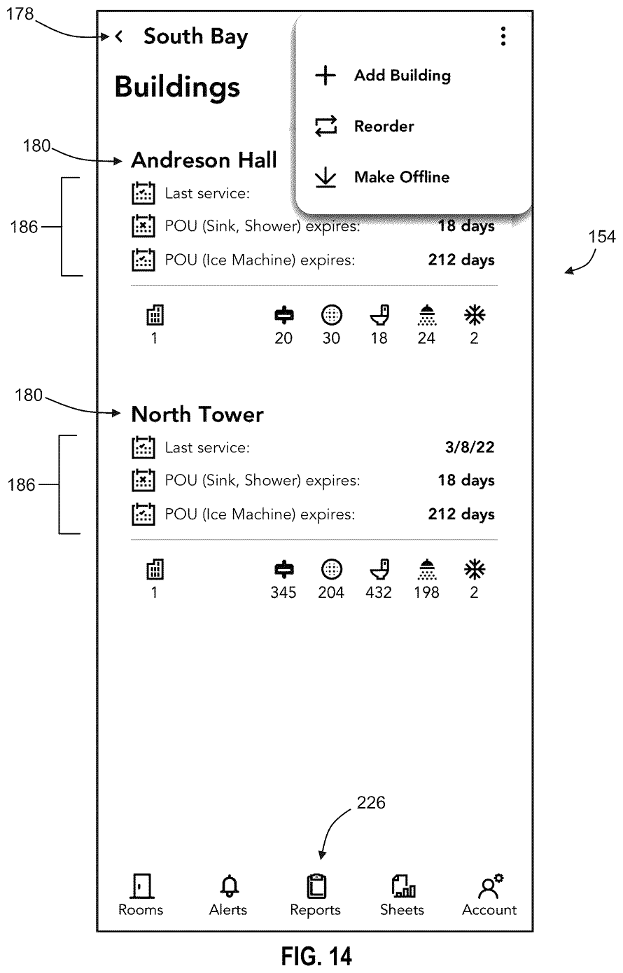

In preferred implementations of the system 100, for each of one or more technician users 132, the server operations 110 may comprise providing one or more control point status display pages 154 for display on the user device 136 of the technician user 132 by way of the authenticated connection 152 (see, e.g., FIGS. 9-14). Each of the control point status display pages 154 may be based on at least a subset (e.g., a portion) of the room data 112 for the rooms 114 corresponding to the location permissions 150 of the technician user 132.

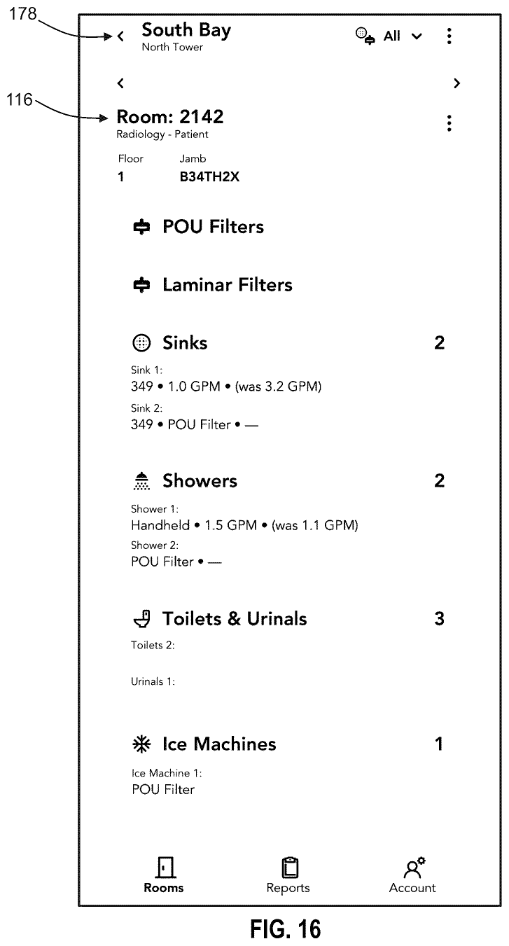

Referring to FIG. 3, in preferred implementations of the system 100, for each of one or more technician users 132, the server operations 110 may comprise, in response to a room edit request from the user device 136 of the technician user 132 for a requested one of the rooms 114 corresponding to the location permissions 150 of the technician user 132, providing a room edit interface 156 for the requested room 114 (see, e.g., FIGS. 16-18). The room edit interface 156 may be configured to enable the technician user 132 to enter service input data 158 pertaining to the requested room 114.

Referring to FIGS. 3 and 16-18, in preferred implementations of the system 100, for each of one or more technician users 132, the server operations 110 may comprise updating room data 112 stored to the server database 104 based on service input data 158 for each room 114 for which service input data 158 is entered.

Referring to FIG. 1, in preferred implementations of the system 100, for each of one or more supervisory users 134, the server operations 110 may comprise maintaining an authenticated connection 160 between the server database 104 and the user device 138 of the supervisory user 134. The authenticated connection 160 may preferably be based upon the login credentials 144 of the supervisory user 134.

Referring to FIGS. 3 and 9-14, in preferred implementations of the system 100, for each of one or more supervisory users 134, the server operations 110 may comprise providing one or more control point status display pages 154 for display on the user device 138 of the supervisory user 134 by way of the authenticated connection 160. Each of the control point status display pages 154 may be based on a subset (e.g., a portion) of the room data 112 for the rooms 114 corresponding to the location permissions 150 of the supervisory user 134.

Referring to FIG. 1, particular preferred implementations of the system 100 are configured to maintain the authenticated connection 152 for multiple technician users 132 simultaneously.

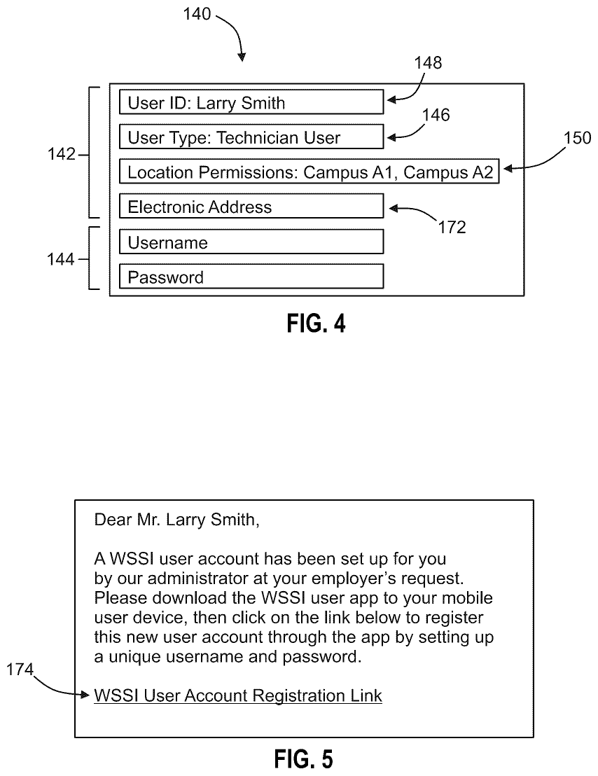

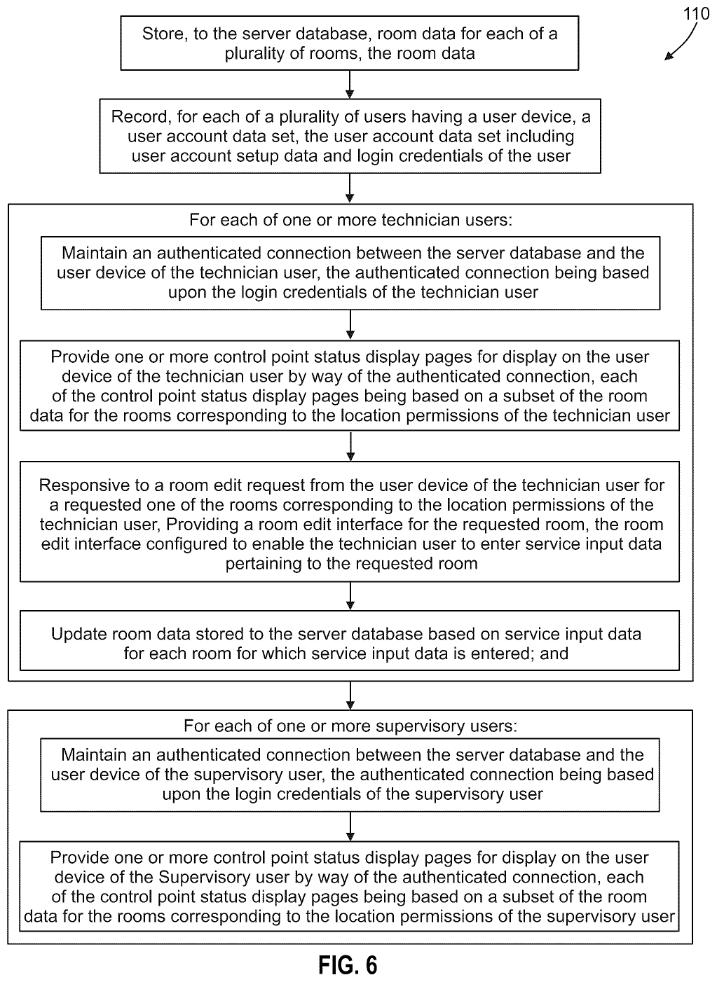

Referring to FIG. 4, in certain preferred implementations of the system 100, the location permissions 150 of one or more technician users 132 may be different from the location permissions 150 of other technician users 132. Similarly, the location permissions 150 of one or more supervisory users 134 may be different from the location permissions 150 of other supervisory users 134.

Referring to FIG. 1, in particular preferred implementations of the system 100, the user device 136 of each technician user 132 may include a user device processor 162 and a user device memory 164. The user device memory 164, which is not a transitory propagating signal, may be configured to be operably connected to the user device processor 162 and may include user device processor executable instructions 166. The user device processor executable instructions 166, when executed by the user device processor 162, cause the user device processor 162 to perform user device operations 168.

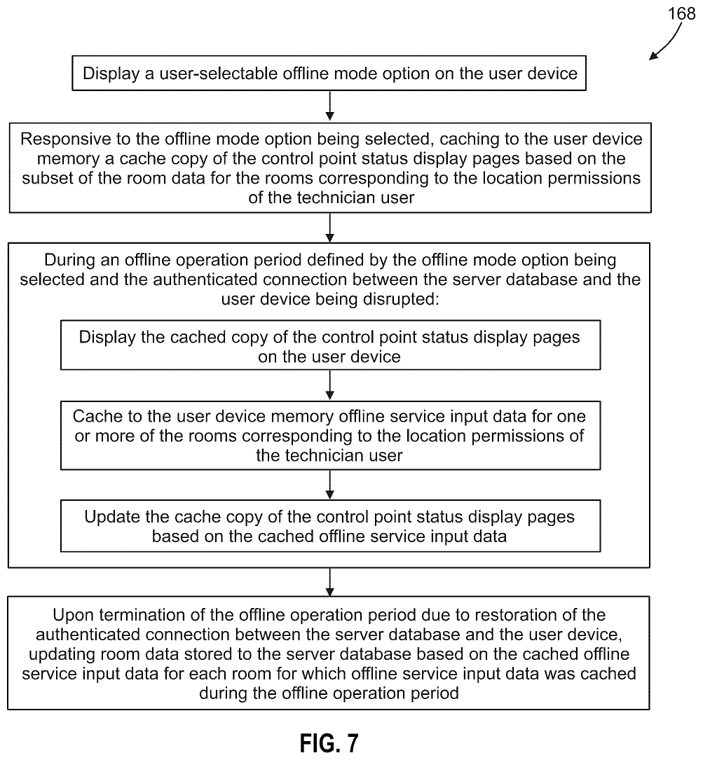

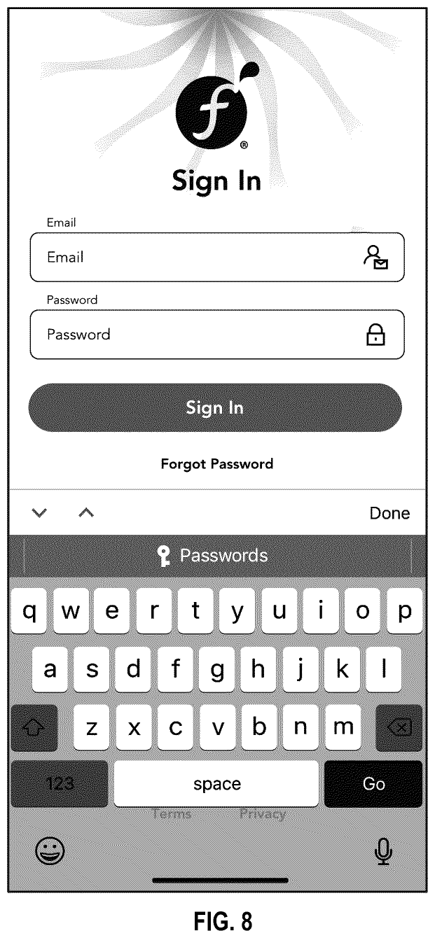

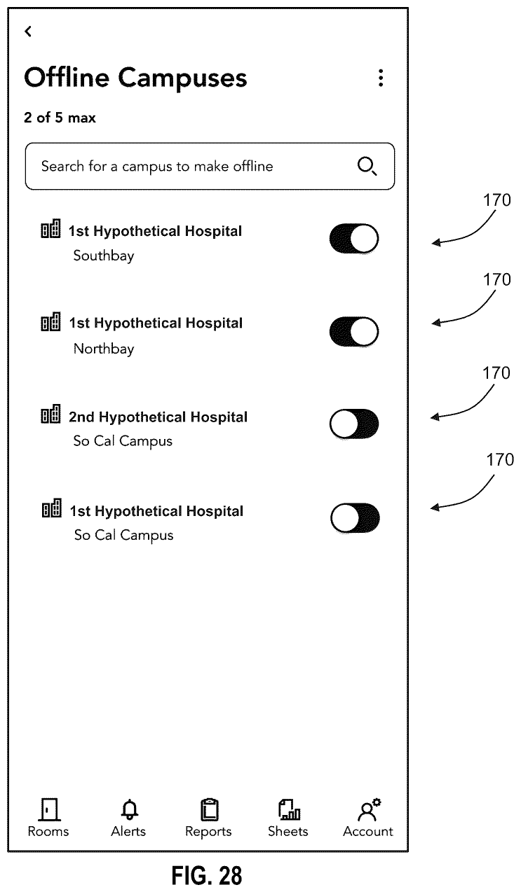

Referring to FIGS. 1, 7 and 28, in particular implementations of the system 100, the user device operations 168 performed by the user device processor 162 may comprise displaying a user-selectable offline mode option 170 on the user device 136. The user device operations 168 may further comprise, in response to the offline mode option 170 being selected, caching to the user device memory 164 a cache copy of the control point status display pages 154 based on the subset of the room data 112 for the rooms 114 corresponding to the location permissions 150 of the technician user 132. Moreover, the user device operations 168 may further comprise, during an offline operation period defined by the offline mode option 170 being selected and the authenticated connection 152 between the server database 104 and the user device 136 being disrupted, displaying the cached copy of the control point status display pages 154 on the user device 136. The user device operations 168 may further comprise caching to the user device memory 164 offline service input data for one or more of the rooms 114 corresponding to the location permissions 150 of the technician user 132. The user device operations 168 may further comprise updating the cache copy of the control point status display pages 154 based on the cached offline service input data. The user device operations 168 may further comprise, upon termination of the offline operation period due to restoration of the authenticated connection 152 between the server database 104 and the user device 136, updating room data 112 stored to the server database 104 based on the cached offline service input data for each room 114 for which offline service input data was cached during the offline operation period.

Referring to FIGS. 4 and 5, in certain preferred implementations of the system 100, for each of the users (e.g., 132, 134), the user account setup data 140 may include an electronic address 172 enabling transmission of a message to the user device (e.g., 136, 138) of the user (e.g., 132, 134). In such implementations, the server operations 110 performed by the server processor 102 may further comprise transmitting, by way of the electronic address 172, an account registration link 174 to the user device (e.g., 136, 138). The account registration link 174 may be configured to enable the respective user (132, 134) to define the login credentials 144 in the respective user account data set 140. The electronic address 172 may be, for example, an email address, an SMS number or the like.

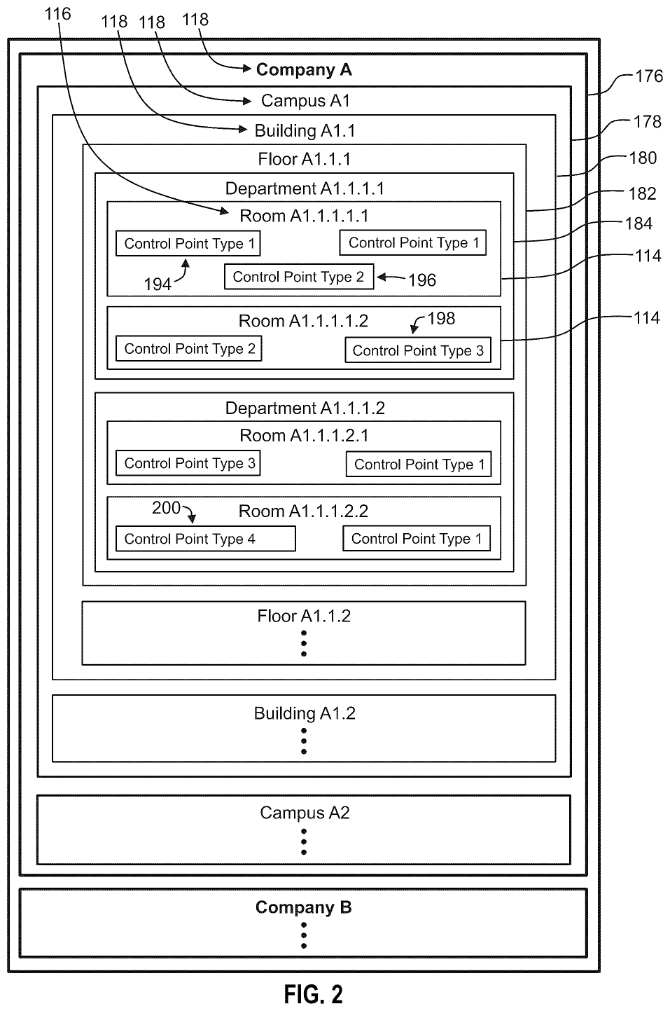

Referring to FIGS. 2 and 3, in particular preferred implementations of the system 100, for each room 114, the location groups 120 may include a building-level group 180 defined at least in part by a building within which the associated room 114 resides.

In certain preferred implementations of the system 100, for each room 114, the location groups 120 may include a company-level group 176 defined at least in part by a company that operates the building within which the associated room 114 resides.

In particular preferred implementations of the system 100, for one or more of the rooms 114, the location groups 120 may include a campus-level group 178 defined at least in part by a campus encompassing the building within which the associated room 114 resides.

In certain preferred implementations of the system 100, for one or more of the rooms 114, the location groups 120 may include a floor-level group 182 defined at least in part by a floor within which the associated room 114 resides, that floor 114 being within the building within which the associated room 114 resides.

In particular preferred implementations of the system 100, for one or more of the rooms 114, the location groups 120 include a department-level group 184 defined at least in part by a department within which the associated room 114 resides.

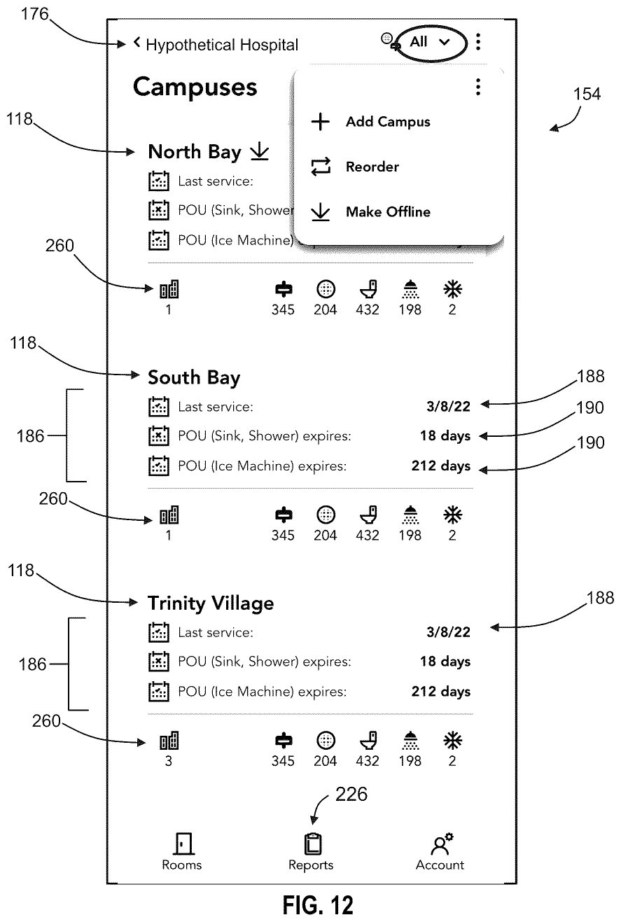

Referring to FIG. 12, in certain preferred implementations of the system 100, for each of the users (e.g., 132, 134), the control point status display pages 154 may be configured to include control point status information 186. The control point status information 186 may preferably be based on room data 112 for all of the rooms 114 associated with a respective location group 120. Moreover, the control point status display pages 154 may be configured to be user-selectable based on location group 120 for display on the user device (e.g., 136, 138).

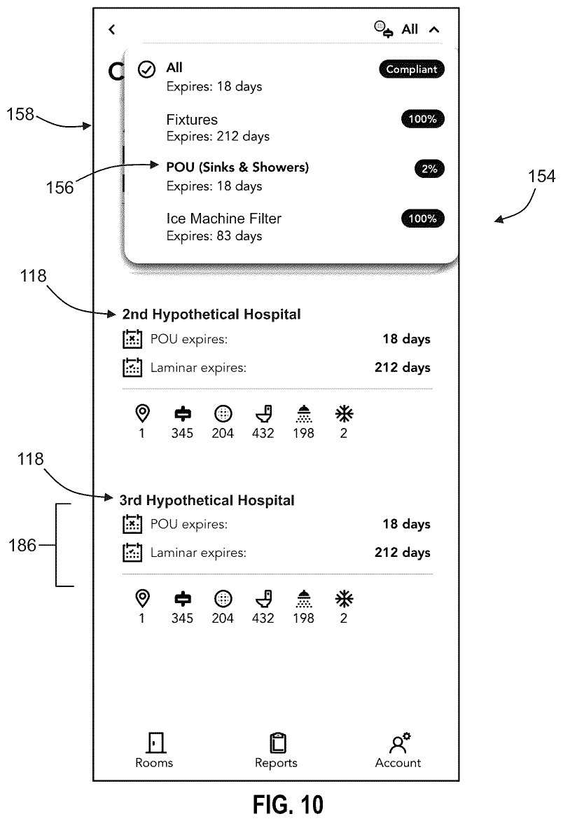

Referring o FIG. 10, a control point status display page 154 may be configured to present expiration indicators, such as a point-of-use (POU) expiration indicator 256 and a fixture expiration indicator 258. A “fixture” may preferably refer to a control point where a laminar or a shower head is to be replaced, for example, every 12 months or similar timeline (e.g., at certain sink faucets and shower heads). In contrast, a “POU” may preferably refer to control points where a filter is to be replaced, for example, every 2 months or similar timeline (e.g., at certain sink faucets and showers). As shown for example in FIG. 10, each expiration indicator may include a corresponding number of days to the first-required replacement due within the respective selected group level or room. Alternatively, or in addition, a percentage or ratio indication may be provided. For example, supervisory users may be presented with a percentage of control point service completions (e.g., control points which have been serviced by a pre-set amount of time in advance of expiration), whereas technician users may be presented with the same information in a ratio format (e.g., 68/68 instead of 100%).

Referring to FIG. 12, in particular preferred implementations of the system 100, for each control point status display page 154 user-selected based on a company-level group 176, the control point status information 186 may include, for each of the campus-level groups 178 corresponding to the company-level group 176, the group identifier 118 and some or all of the following (in no particular order): (a) the date of last service completion 188 for the most recently-serviced room 114 associated with the campus-level group 178, (b) for at least one type of water control point 122 within any room 114 associated with the campus-level group 178, a service expiration day indication 190, and (c) a total of the quantity 192 of each type of water control point 122 within all rooms 114 associated with the campus-level group 178. The service expiration day indication 190 may be calculated, for example, by adding the prescribed service interval 124 for the type of water control point 122 to the oldest service completion date of any room 114 associated with the campus-level group 178 that includes the respective type of water control point 122.

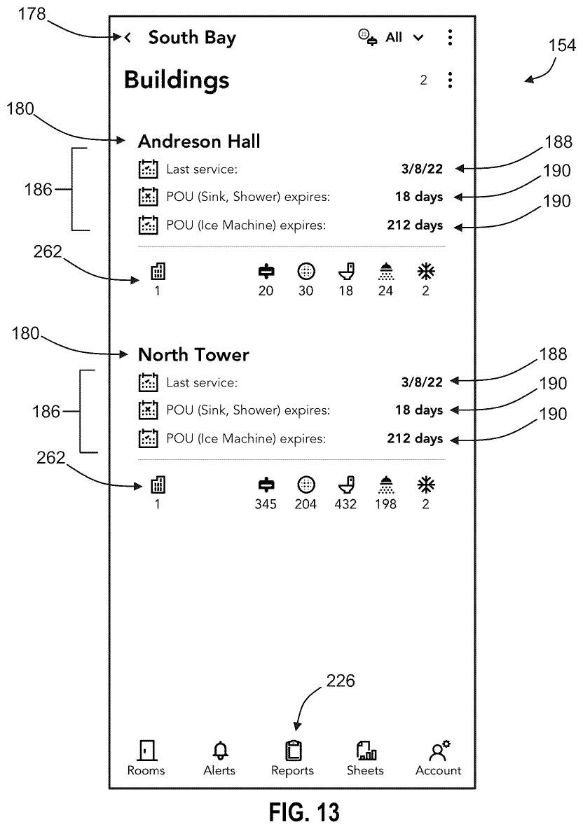

Referring to FIG. 13, in certain preferred implementations of the system 100, for each control point status display page 154 user-selected based on a campus-level group 178, the control point status information 186 may include, for each of the building-level groups 180 corresponding to the campus-level group 178, the group identifier 118 and some or all of the following (in no particular order): (a) the date of last service completion 188 for the most recently-serviced room 114 associated with the building-level group 180, (b) for at least one type of water control point 122 within any room 114 associated with the building-level group 180, a service expiration day indication 190, and (c) a total of the quantity 192 of each type of water control point 122 within all rooms 114 associated with the building-level group 178. The service expiration day indication 190 may be calculated, for example, by adding the prescribed service interval 124 for the type of water control point 122 to the oldest service completion date of any room 114 associated with the building-level group 180 that includes the respective type of water control point 122.

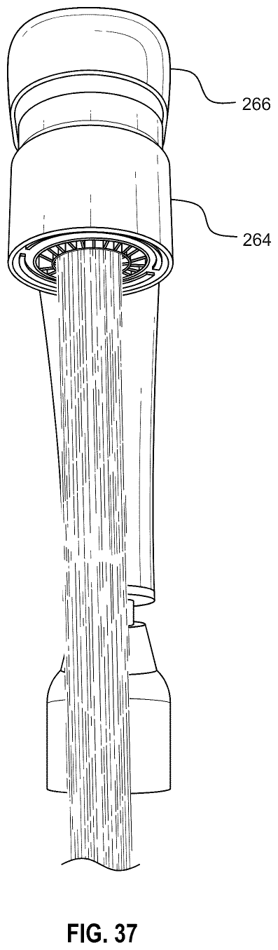

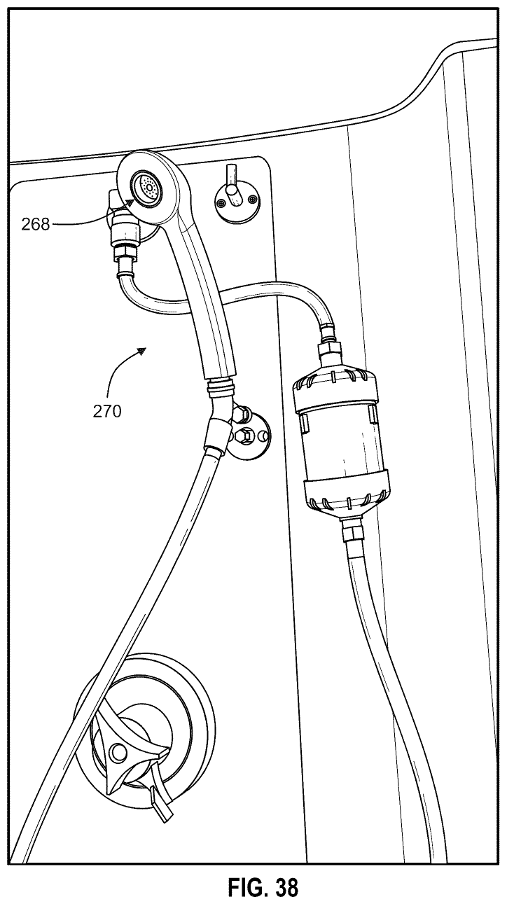

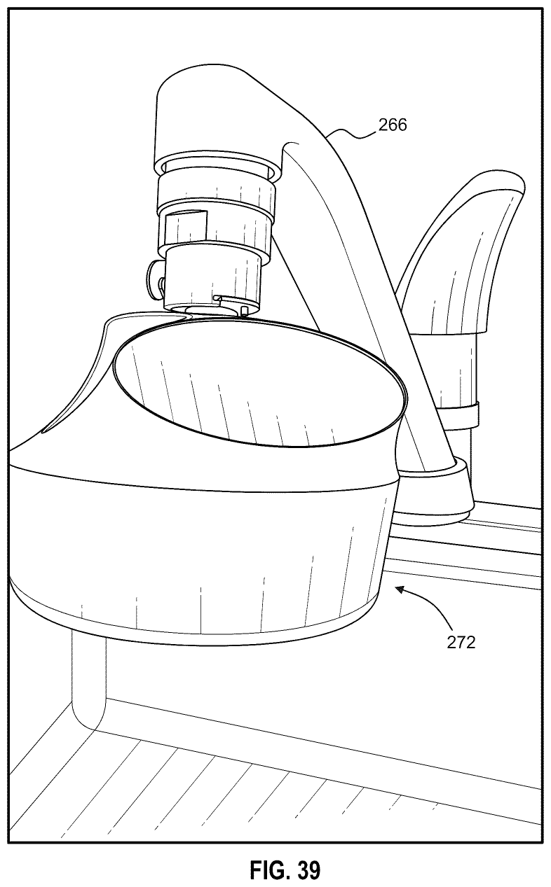

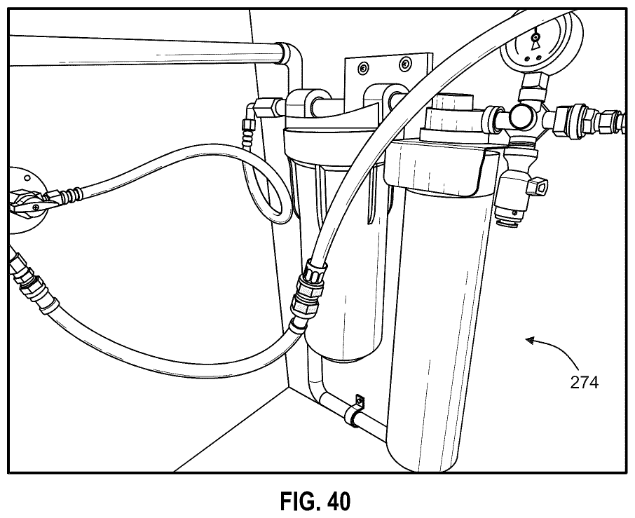

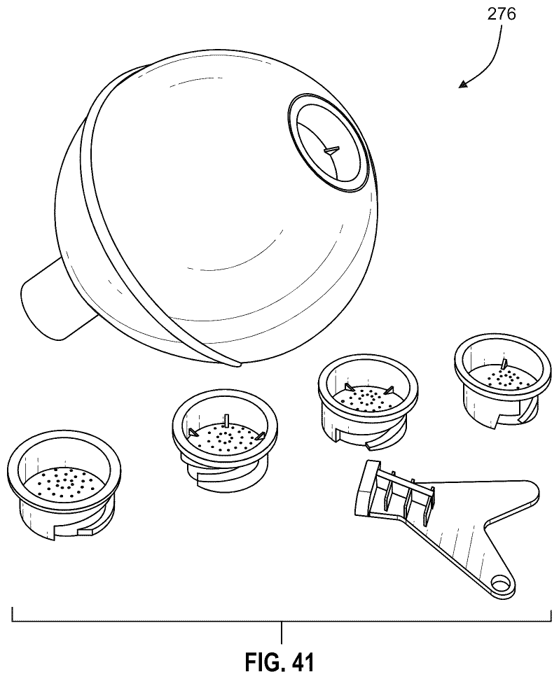

In particular preferred implementations of the system 100, each type of water control point 122 may be selected from the group consisting of a first control point type 194, a second control point type 196, a third control point type 198 and a fourth control point type 200. Each first control point type 194 may correspond to, for example, a sink laminar flow device (see, e.g., FIG. 37). Each second control point type 196 may correspond to, for example, a sink filter or a shower filter (see, e.g., FIG. 39). Each third control point type 198 may correspond to, for example, a showerhead (see, e . . . , FIG. 38), and each fourth control point type 200 may correspond to, for example, an ice machine filter (see, e.g., FIG. 40).

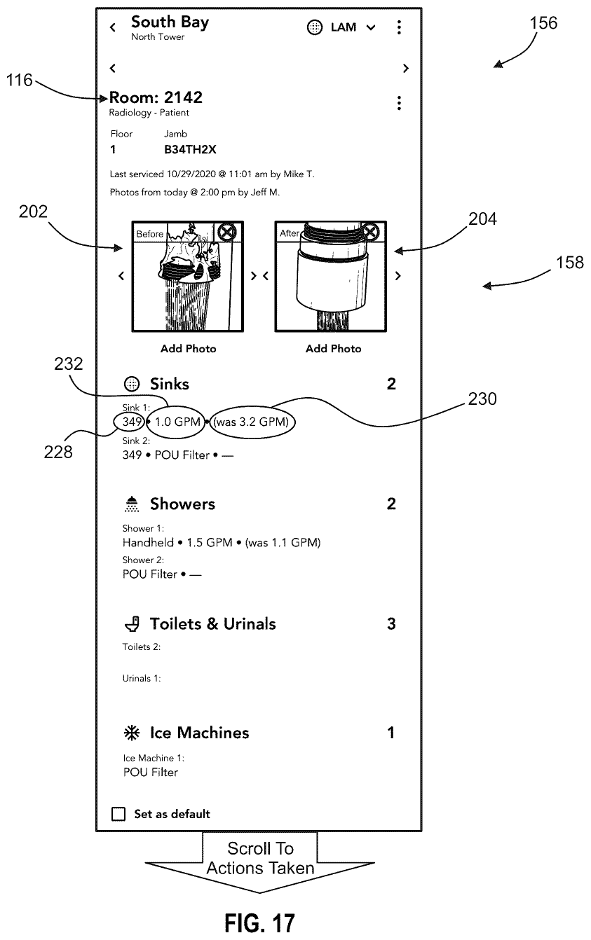

Referring to FIG. 17, in certain preferred implementations of the system 100, the room data 112 for one or more of the rooms 114 may include before-service photos 202 and after-service photos 204. In such implementations, the service input data 158 may include a before-service photo 202 and an after-service photo 204 corresponding to one or more of the water control points serviced by the user technician in the respective room 114.

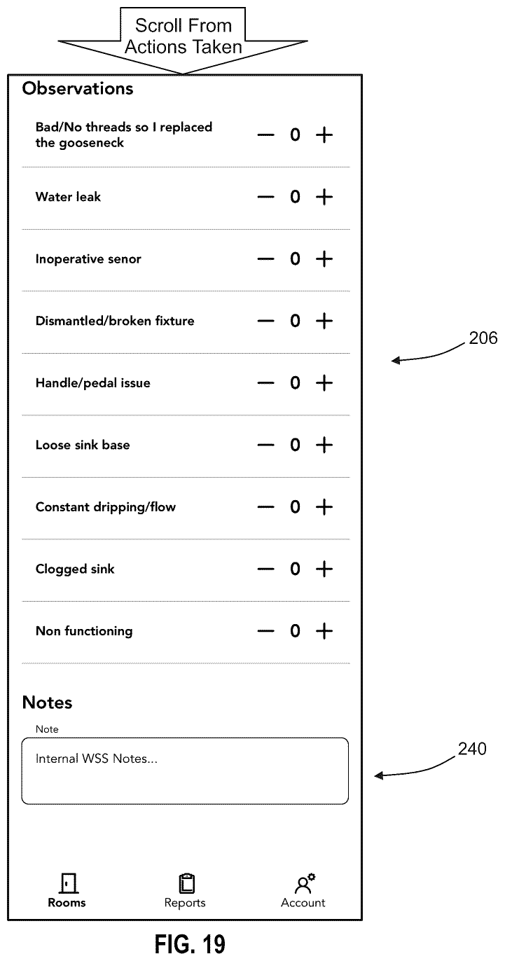

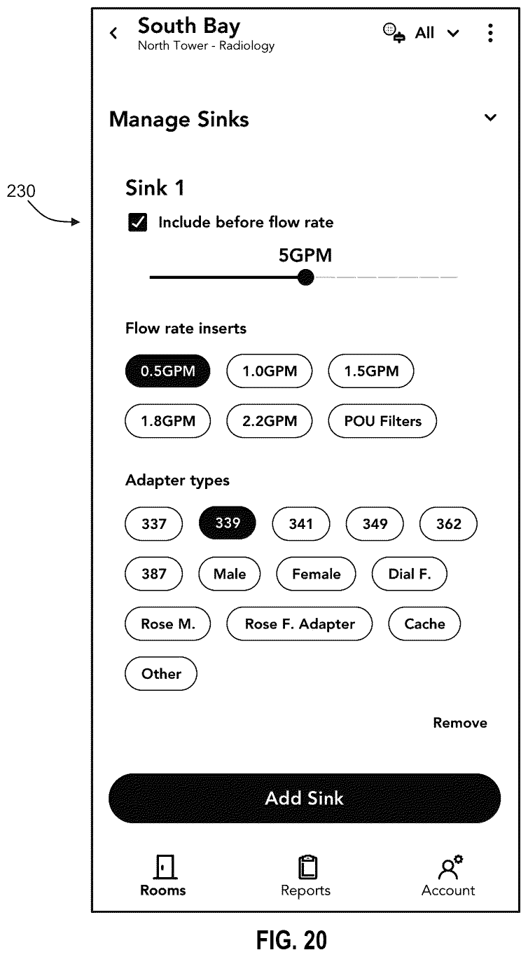

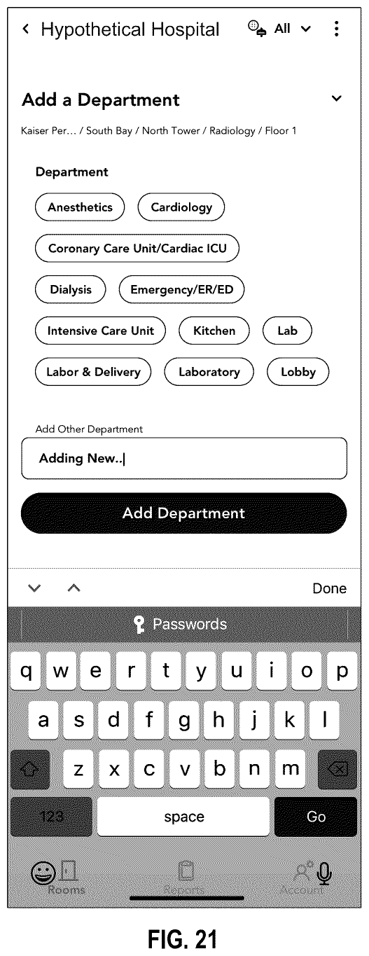

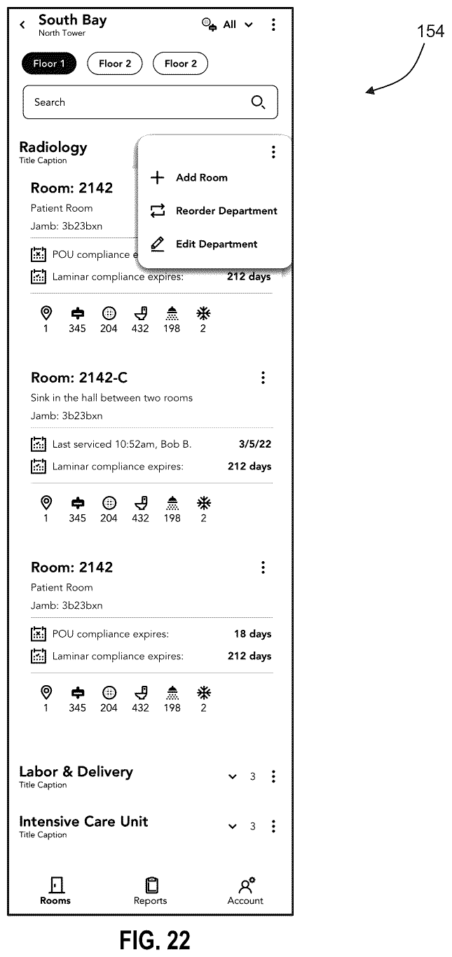

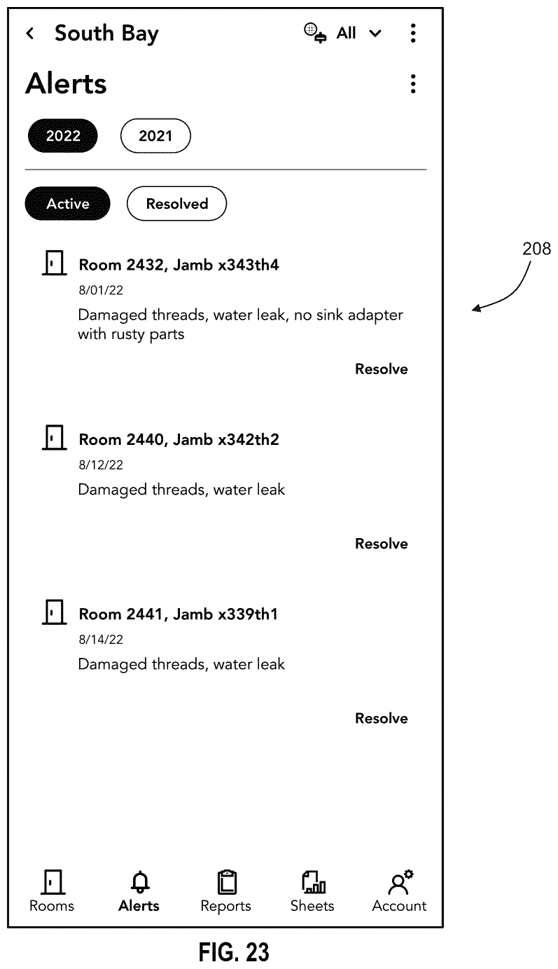

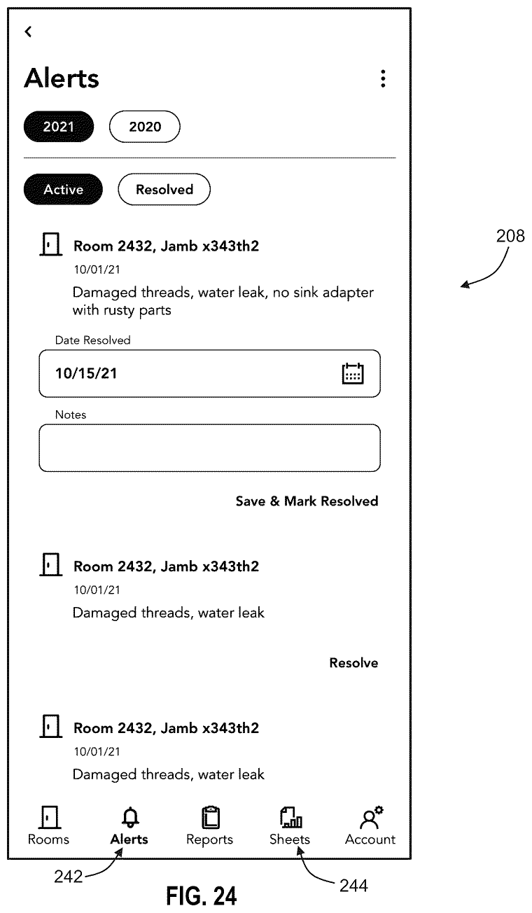

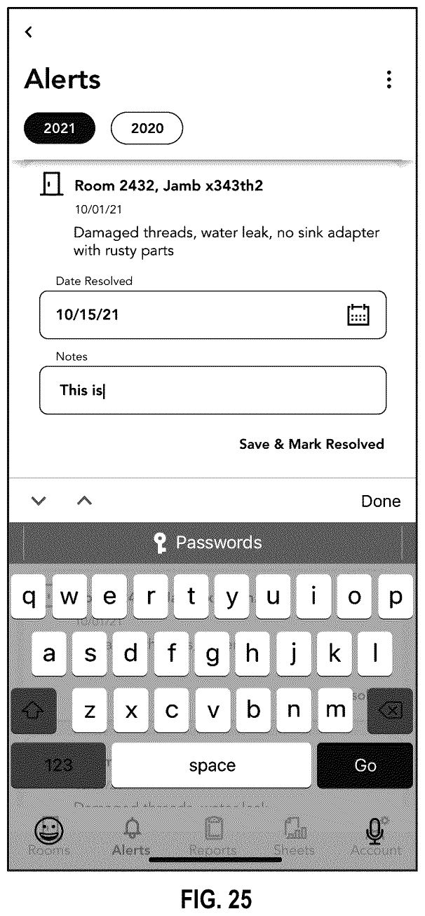

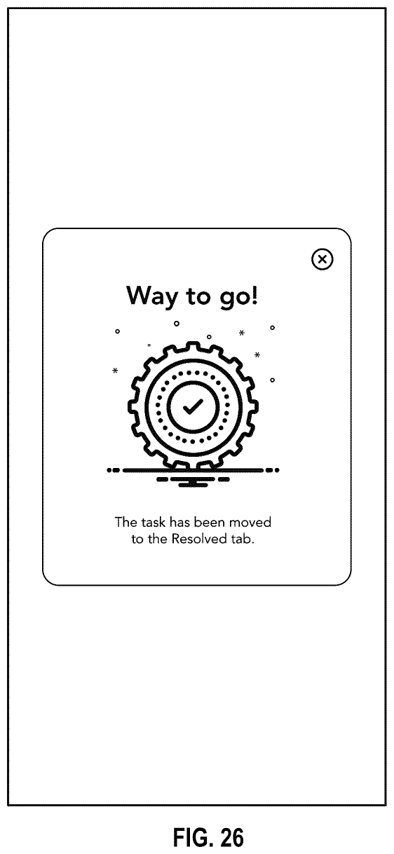

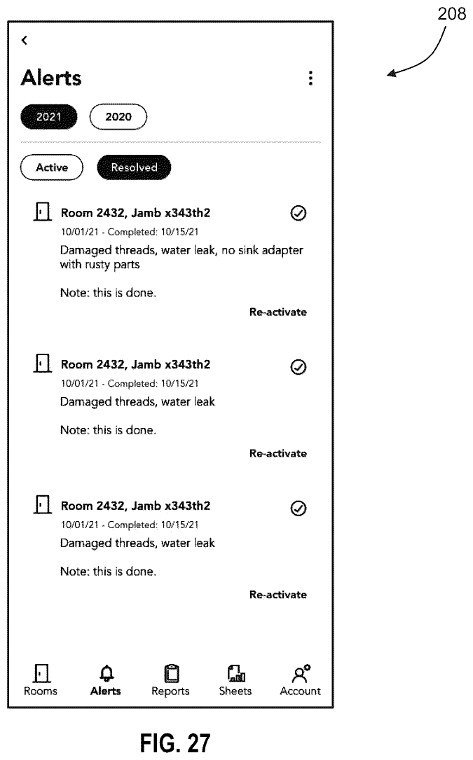

Referring to FIG. 19, in particular preferred implementations of the system 100, the service input data 158 may include observation data 206 input by the technician user 132 in connection with an observed one of the rooms 114 corresponding to the location permissions 150 of the technician user 132. In such implementations, the server operations performed by the server processor 102 may further comprise conveying, by authenticated connection 160, the observation data to a user device 138 of a supervisory user 134 having location permissions 150 corresponding to the observed room 114. This observation conveyance mechanism allows a technician user to alert a supervisory user of certain issues outside of the expertise and qualifications of user technician that may require the attention of, for example, a certified plumber. Referring to FIG. 23, the supervisory user 134 may receive the observation data, on, for example, an alerts display user 134 may also have an option to indicate, by way of the alerts page 208, that the alerts have been resolved on their end. For example, with reference to FIG. 24, once the supervisory user has had a certified plumber resolve a plumbing issue identified by the user technician, that supervisory user can enter this alert resolution data to confirm that the alert has been resolved. In preferred embodiments of the system 100, the user technician does not enter this alert resolution data. A corresponding alert resolution indication may then be, for example, received by the server database 104 for viewing by an administrator by way of the administrator portal 210. Referring again to FIG. 24, a sheets icon 244 may be presented on, for example, an alerts display page 208 of a supervisory user. In certain preferred embodiments of the system 100, only a supervisory user type would be presented with the sheets icon 244. Selecting of the sheets icon 244 may be configured to cause display of a list of clickable PDFs with, for example, cut sheets of manufacturer specs associated with the installed filter or laminar at the respective control point.

Referring to FIGS. 1 and 3, in certain preferred implementations of the system 100, the server operations 110 performed by the server processor 102 may further comprise providing an administrator portal 210 configured to establish an authenticated administrator connection 212 between the server database 104 and an administrator device 214 of an administrator 216. In such implementations, for each user 114, the server operations 110 performed by the server processor 102 may further comprise obtaining the user account setup data 142 by way of the administrator portal 210 before the server operation of recording, for each of the users (e.g., 132, 134), the user account data set 140.

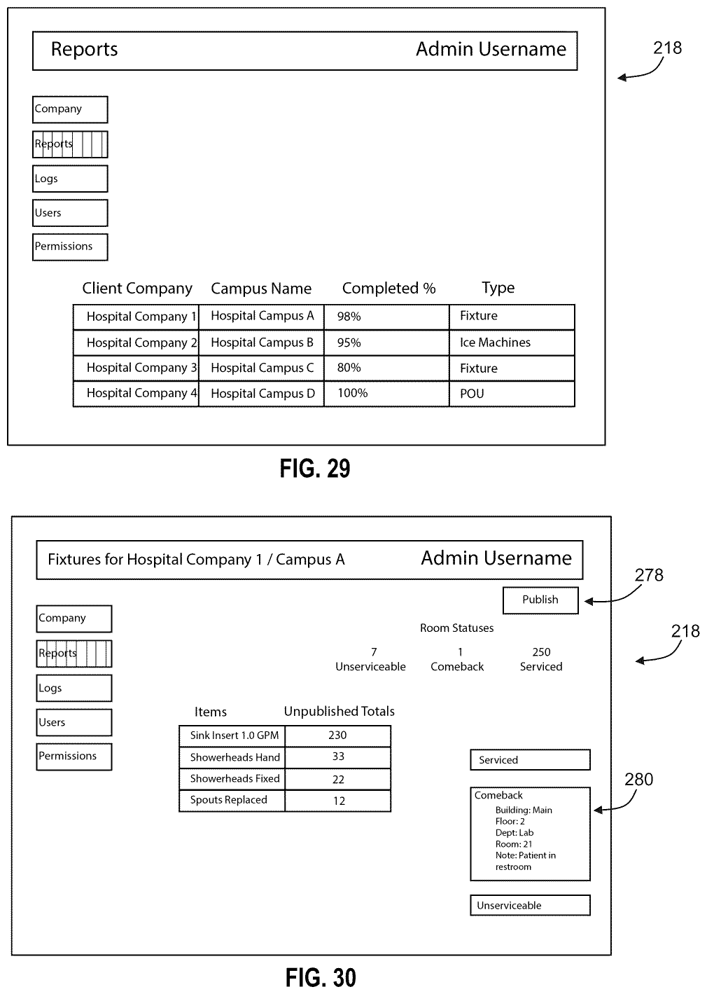

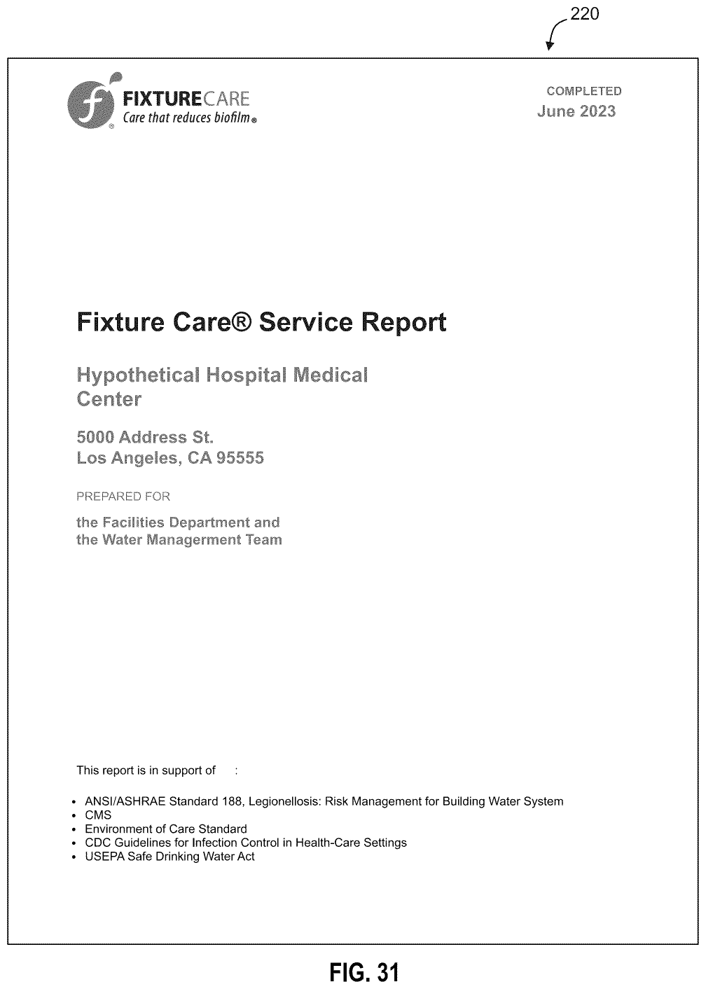

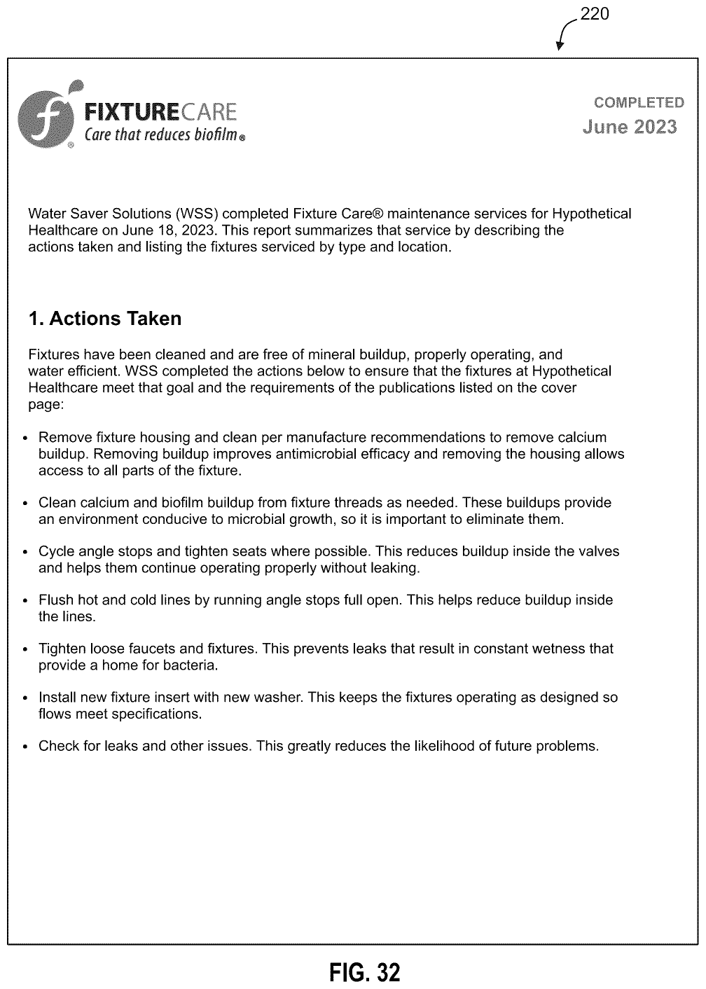

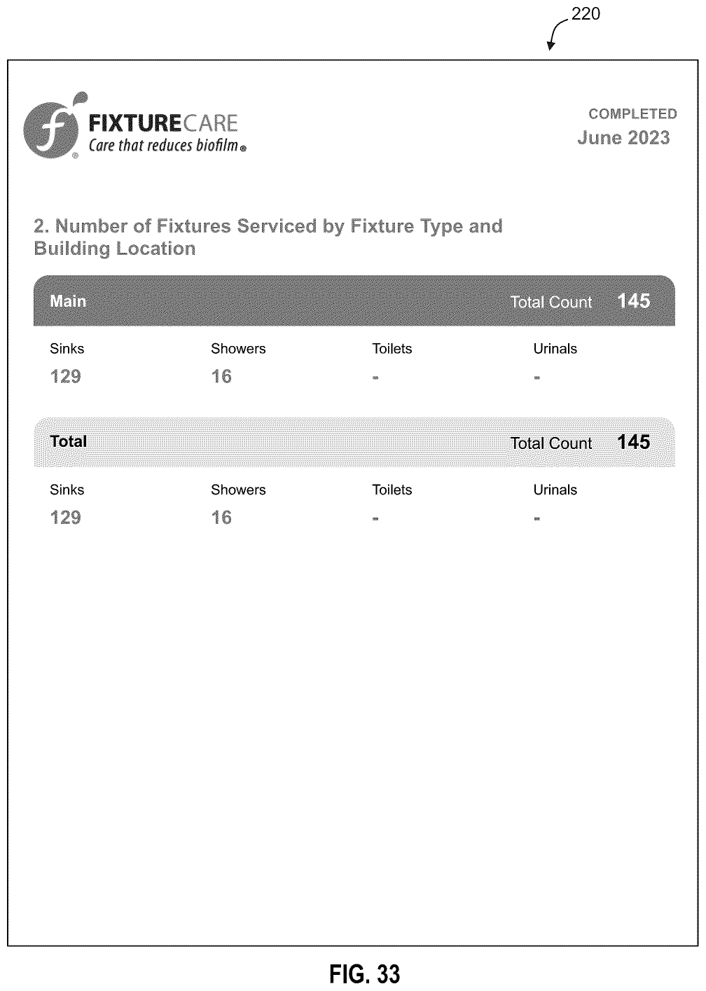

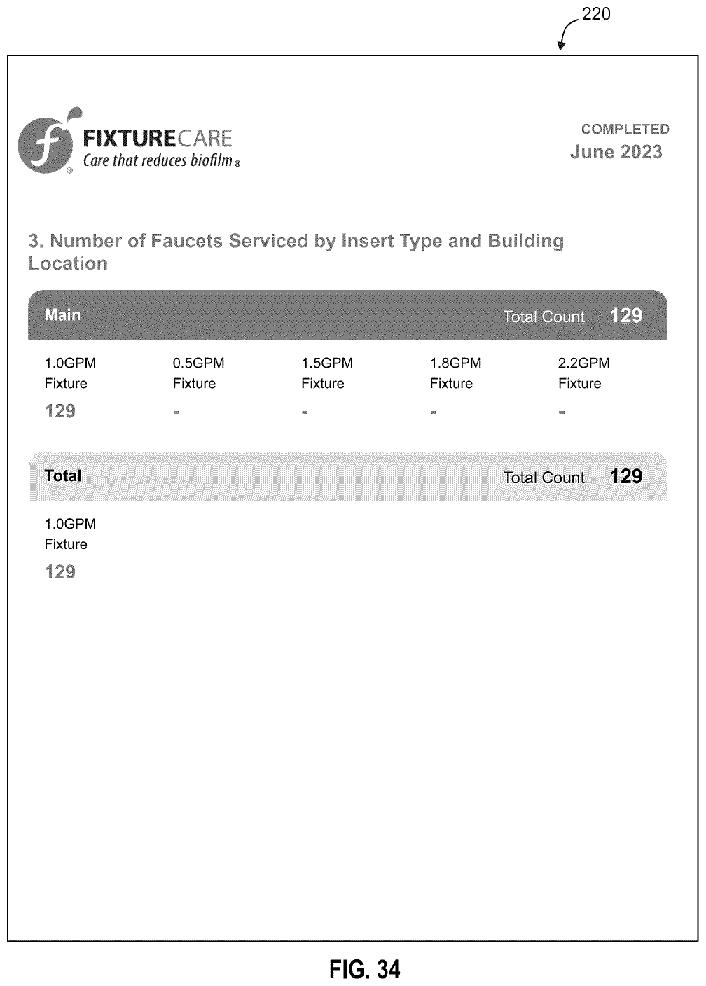

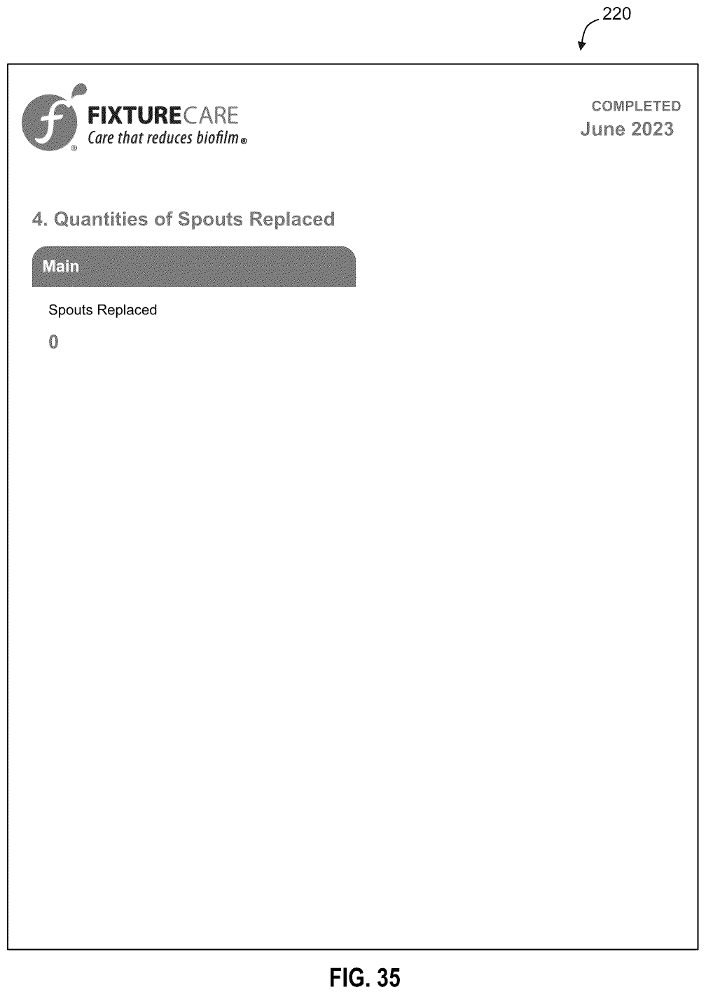

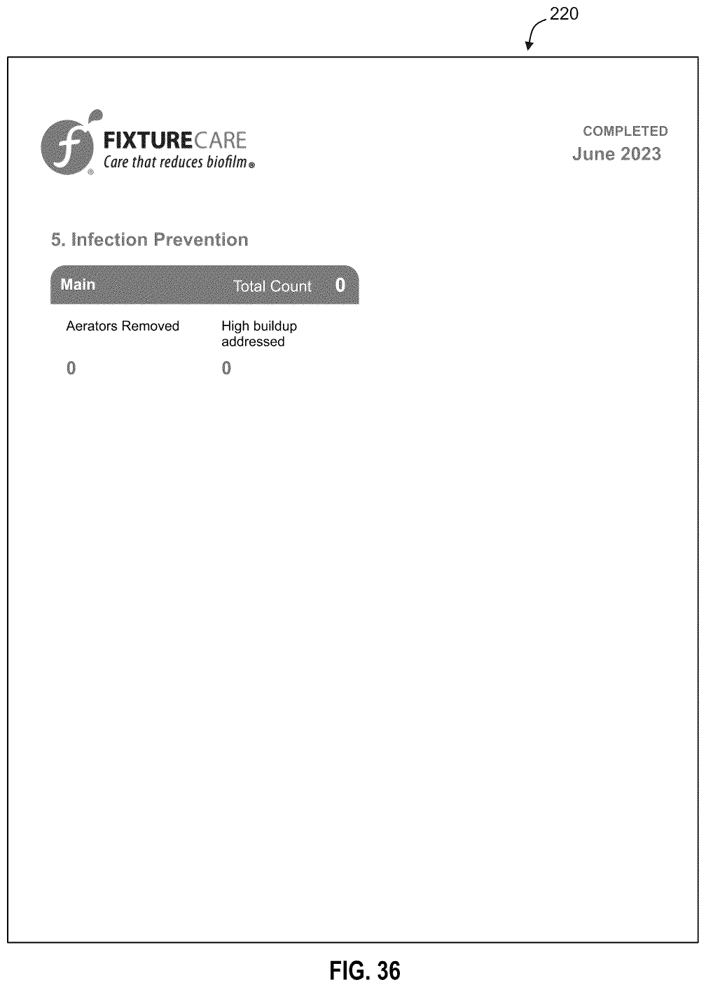

Referring to FIGS. 3, 29 and 30, in particular preferred implementations of the system 100, the server operations 110 performed by the server processor 102 may further comprise providing a report publication interface 218 by way of the administrator portal 210, and generating, for each campus-level group 178 by way of the report publication interface 218, a service report 220 (see, e.g., FIGS. 31-36). The service report 220 may preferably include, for example, one or more of the following (in no particular order): (a) the group identifier 118, (b) a campus service completion date 222, (c) identification of the types of water control points 122 within all rooms 114 associated with the campus-level group 178, and (d) for each of the types of water control points 122 within rooms 114 associated with the campus-level group 178, a tally 224 of the water control points for which service has been completed as of the service completion date. The server operations 110 performed by the server processor 102 may further comprise including, on one or more of the control point status display pages 154, a user-selectable report icon 226. Responsive to a supervisory user 134 selecting the report icon 226, the supervisory user device 138 would be provided access to the service reports 220 only for campus-level groups 178 associated with rooms 114 corresponding to the location permissions 150 of the icon-selecting supervisory user 134. The service reports 220 may preferably provided in PDF format, or a similar format which may be viewable with a conventional document viewer (e.g., conventional PDF viewer) on a mobile user device.

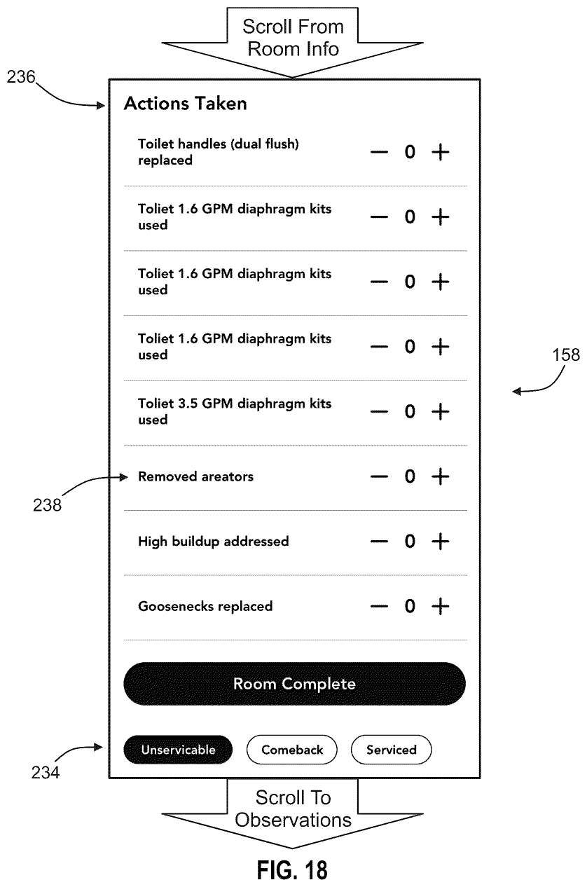

Referring to FIG. 18, in certain preferred implementations of the system 100, the service input data 158 may include a user-selectable room status. The user-selectable room status options 234 may preferably be unserviceable, comeback and serviced. If a particular technician has serviced all the water control points in the room, they would then select the “Serviced” icon and select “Room Complete.” In contrast, if there is something temporarily preventing the technician user from servicing one or more of the control points in the room, they may select “Comeback.” In further contrast, if there is something that appears to be permanently preventing the technician user from servicing all water control points within the room (e.g., a water control point is blocked by a large machine like an MRI), they may select “Unserviceable.” In particular implementations, the administrator portal 210 may be configured to enable the administrator 216 to review and modify the room status entered by the technician user.

Referring to FIG. 19, in the observation data page of the room edit interface, a notes entry field 240 may be provided. Any notes entered by the technician user may be sent exclusively to the administrator to facilitate their decision-making and to resolve issues beyond the purview and expertise of the technician user. For example, the administrator may reach out to the supervisory user (or their company) to resolve a non-standard issue raised by a technician user, or may remove a control point from consideration, etc.

In some embodiments of the system 100, various application designs may be implemented using various technologies. In an illustrative example, some embodiment functions may be split across a Web Application and Mobile Application. Some Web Application embodiment designs may run on the Linux® Operating System backed by MySQL® storage access implemented with programming language and supporting frameworks including PHP®, Angular JS, Angular Bootstrap, JQUERY®, HTML 5, CSS®, and JS® (JAVASCRIPT®). Various embodiment web application designs may be provided via AWS® by an APACHE® Web Server hosted in an AMAZON® CLOUD® production environment. Some mobile application embodiments may be implemented across multiple platforms, for example IOS® and ANDROID®, using programming languages including JAVASCRIPT®, SWIFT®, JAVA®, and OBJECTIVE-C®, with supporting frameworks such as React Native.

In the present disclosure, various features may be described as being optional, for example, through the use of the verb “may;”, or, through the use of any of the phrases: “in some embodiments,” “in some implementations,” “in some designs,” “in various embodiments,” “in various implementations,”, “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments, elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)−(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects may lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

According to an embodiment of the present invention, the system and method may be accomplished through the use of one or more computing devices. As depicted, for example, at least in FIG. 1, one of ordinary skill in the art would appreciate that an exemplary system appropriate for use with embodiments in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with embodiments of the present invention include, but are not limited to, proprietary computing devices, personal computers, mobile computing devices, tablet PCs, mini-PCs, servers or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In various embodiments, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with embodiments of the present invention, and embodiments of the present invention are contemplated for use with any appropriate configuration.

According to an embodiment of the present invention, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth connections, or any combination thereof. One of ordinary skill in the art would appreciate that there are numerous communications means that may be utilized with embodiments of the present invention, and embodiments of the present invention are contemplated for use with any communications means.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general purpose hardware and computer instructions; and so on-any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a sequence of computational instructions or program instructions. It will be appreciated that a programmable apparatus (i.e., computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these.

All such implementations are within the scope of the present disclosure. Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

The following listing matches certain terminology used within this disclosure with corresponding reference numbers used in the non-limiting examples illustrated in the several figures.
100 system
101 server (i.e., water control point care platform; e.g., WCPC platform)
102 server processor
104 server database
106 server memory
108 server processor executable instructions
110 server operations
112 room data
114 room
116 room identifier
118 group identifier (e.g., company-level, campus-level, building-level, floor-level)
120 location group
122 water control point types
124 prescribed service interval (e.g., 2 months, 6 months, 12 months, etc.)
126 quantity
128 service completion status
130 date of last service completion
132 technician user
134 supervisory user
136 technician user device
138 supervisory user device
140 user account data set
142 user account setup data
144 login credentials
146 user type
148 user identity (e.g., the name of a user)
150 location permissions
152 authenticated connection (between server database and the technician user device)
154 control point status display page
156 room edit interface
158 service input data
160 authenticated connection (between server database and the supervisory user device)
162 user device processor
164 user device memory
166 user device processor executable instructions
168 user device operations
170 user-selectable offline mode option
172 electronic address (e.g., email address or SMS number)
174 account registration link
176 company-level group
178 campus-level group
180 building-level group
182 floor-level group
184 department-level group
186 control point status information
188 date of last service completion
190 service expiration date indication (e.g., in date format or as number of days to expiration)
192 total of quantity of water control point type
194 first control point type (e.g., sink laminar flow device)
196 second control point type (e.g., sink filter or a shower filter)
198 third control point type (e.g., showerhead)
200 fourth control point type (e.g., ice machine filter)
202 before-service photo
204 after-service photo
206 observation data (Note: these observations may preferably be immediately pushed to the user device (in the WSSI app) of supervisory user)
208 alerts display page
210 administrator portal
212 authenticated administrator connection
214 administrator device
216 administrator
218 report publication interface
220 service report
222 campus service completion date
224 tally of water control points
226 report icon
228 adapter type
230 initial control point flow rate (e.g., entered by technician user once, before a filter or laminar is installed by that technician user at the control point)
232 manufacturer-specified flow rate (post-laminar/filter installation flow rate)
234 technician user-selectable room status options
236 actions taken (e.g., input by technician user for specific room; e.g., may preferably be displayed on the service report, and may preferably not be immediately pushed to a supervisory user)
238 removed aerators entry field (Note: Aerators are no longer allowed in hospital due to Legionnaires disease. So this aspect allows a record to be kept that verifies that an aerator has been removed (allows the customer to demonstrate compliance).
240 notes entry field (e.g., the notes entered by preferably only go to admin portal)
242 alerts icon (e.g., may only be viewable/selectable by a supervisory user)
244 sheets icon
246 laminar icon (with number indicating total sink laminars installed throughout the respective group level or room)
248 filter icon (with number indicating total sink and shower filters installed throughout the respective group level or room)
250 ice machine icon (with number indicating total ice machine filters installed throughout the respective group level or room)
252 campus icon (with number indicating total campuses within the respective company level)
254 shower icon (with number indicating total replaceable shower heads within the respective group level or room)
256 point of use expiration indicator (e.g., indicating the nearest date a sink or shower filter within the respective group level or room will need to be replaced)
258 fixture expiration indicator (e.g., indicating the nearest date a laminar or shower head within the respective group level or room will need to be replaced)
260 building icon (e.g., with number indicating total buildings with the respective campus)
262 floor icon (e.g., with number indicating total floors with the respective building)
264 sink laminar flow device
266 sink faucet
268 removable shower head
270 shower
272 sink filter
274 ice machine filter
276 replacement shower head assembly
278 publish button (e.g., only selectable once all rooms are complete (no comeback statuses))
280 comeback indication (e.g., indicating that comeback needs to be addressed before publish button can be selected)
310 network cloud

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system for facilitating healthcare water control point maintenance and compliance, the system comprising:
a server processor;
a server database, operably coupled with the server processor; and
a server memory that is not a transitory propagating signal, the server memory configured to be operably connected to the server processor and including server processor executable instructions, wherein the server processor executable instructions, when executed by the server processor, cause the server processor to perform server operations comprising:
storing, to the server database, room data for each of a plurality of rooms, the room data including
a room identifier;
group identifiers of location groups with which the room is associated;
types of water control points within the room;
prescribed service interval for each of the types of water control points;
quantity of each type of water control point within the room;
service completion status; and
date of last service completion;
recording, for each of a plurality of users having a user device, a user account data set, the user account data set including user account setup data and login credentials of the user, the user account setup data including a user type, a user identity and location permissions, the user type being either technician user or supervisory user, the location permissions corresponding to one or more of the rooms;
for each of one or more technician users,
maintaining an authenticated connection between the server database and the user device of the technician user, the authenticated connection being based upon the login credentials of the technician user;
providing one or more control point status display pages for display on the user device of the technician user by way of the authenticated connection, each of the control point status display pages being based on a subset of the room data for the rooms corresponding to the location permissions of the technician user;
in response to a room edit request from the user device of the technician user for a requested one of the rooms corresponding to the location permissions of the technician user, providing a room edit interface for the requested room, the room edit interface configured to enable the technician user to enter service input data pertaining to the requested room; and
updating room data stored to the server database based on service input data for each room for which service input data is entered; and
for each of one or more supervisory users,
maintaining an authenticated connection between the server database and the user device of the supervisory user, the authenticated connection being based upon the login credentials of the supervisory user; and
providing one or more control point status display pages for display on the user device of the supervisory user by way of the authenticated connection, each of the control point status display pages being based on a subset of the room data for the rooms corresponding to the location permissions of the supervisory user.

2. The system of claim 1, wherein the system is configured to maintain the authenticated connection for multiple technician users simultaneously.

3. The system of claim 1, wherein
the location permissions of one or more technician users are being different from the location permissions of other technician users; and
the location permissions of one or more supervisory users are being different from the location permissions of other supervisory users.

4. The system of claim 3, wherein the user device of each technician user includes
a user device processor; and
a user device memory that is not a transitory propagating signal, the user device memory configured to be operably connected to the user device processor and including user device processor executable instructions, wherein the user device processor executable instructions, when executed by the user device processor, cause the user device processor to perform user device operations comprising:
displaying a user-selectable offline mode option on the user device;
in response to the offline mode option being selected, caching to the user device memory a cache copy of the control point status display pages based on the subset of the room data for the rooms corresponding to the location permissions of the technician user; and
during an offline operation period defined by the offline mode option being selected and the authenticated connection between the server database and the user device being disrupted,
displaying the cached copy of the control point status display pages on the user device;
caching to the user device memory offline service input data for one or more of the rooms corresponding to the location permissions of the technician user; and
updating the cache copy of the control point status display pages based on the cached offline service input data.

5. The system of claim 4, wherein the user device operations performed by the user device processor further comprise
upon termination of the offline operation period due to restoration of the authenticated connection between the server database and the user device, updating room data stored to the server database based on the cached offline service input data for each room for which offline service input data was cached during the offline operation period.

6. The system of claim 3, wherein for each of the users,
the user account setup data includes an electronic address enabling transmission of a message to the user device of the user; and
the operations performed by the server processor further comprise transmitting, by way of the electronic address, an account registration link to the user device, the account registration link being configured to enable the user to define the login credentials in the user account data set.

7. The system of claim 6, wherein the electronic address is an email address or an SMS number.

8. The system of claim 3, wherein for each room, the location groups include a building-level group defined at least in part by a building within which the associated room resides.

9. The system of claim 8, wherein for each room, the location groups include a company-level group defined at least in part by a company that operates the building within which the associated room resides.

10. The system of claim 9, wherein for one or more of the rooms, the location groups include a campus-level group defined at least in part by a campus encompassing the building within which the associated room resides.

11. The system of claim 10, wherein for one or more of the rooms, the location groups include a floor-level group defined at least in part by a floor within which the associated room resides, the floor being within the building within which the associated room resides.

12. The system of claim 11, wherein for one or more of the rooms, the location groups include a department-level group defined at least in part by a department within which the associated room resides.

13. The system of claim 12, wherein for each of the users, the control point status display pages
are configured to include control point status information based on room data for all of the rooms associated with a respective location group; and
are configured to be user-selectable based on location group for display on the user device.

14. The system of claim 13, wherein for each control point status display page user-selected based on a company-level group, the control point status information includes
for each of the campus-level groups corresponding to the company-level group, the group identifier;
the date of last service completion for the most recently-serviced room associated with the campus-level group;
for at least one type of water control point within any room associated with the campus-level group,
a service expiration day indication calculated by adding the prescribed service interval for the type of water control point to the oldest service completion date of any room associated with the campus-level group that includes the type of water control point; and
a total of the quantity of each type of water control point within all rooms associated with the campus-level group.

15. The system of claim 14, wherein for each control point status display page user-selected based on a campus-level group, the control point status information includes
for each of the building-level groups corresponding to the campus-level group, the group identifier;
the date of last service completion for the most recently-serviced room associated with the building-level group;
for at least one type of water control point within any room associated with the building-level group,
a service expiration day indication calculated by adding the prescribed service interval for the type of water control point to the oldest service completion date of any room associated with the building-level group that includes the type of water control point; and
a total of the quantity of each type of water control point within all rooms associated with the building-level group.

16. The system of claim 1, wherein the room identifier is a room number, a jamb number or a room location description.

17. The system of claim 1, wherein each type of water control point is selected from the group consisting of a first control point type, a second control point type, a third control point type and a fourth control point type.

18. The system of claim 17, wherein
each first control point type corresponds to a sink laminar flow device;
each second control point type corresponds to a sink filter or a shower filter;
each third control point type corresponds to a showerhead; and
each fourth control point type corresponds to an ice machine filter.

19. The system of claim 18, wherein
the room data for one or more of the rooms includes before-service photos and after-service photos; and
the service input data includes a before-service photo and an after-service photo corresponding to one or more of the water control points in the respective room.

20. The system of claim 1, wherein
the service input data includes observation data input by the technician user in connection with an observed one of the rooms corresponding to the location permissions of the technician user; and
the server operations performed by the server processor further comprise conveying, by authenticated connection, the observation data to a user device of a supervisory user having location permissions corresponding to the observed room.

21. The system of claim 1, wherein the server operations performed by the server processor further comprise
providing an administrator portal configured to establish an authenticated administrator connection between the server database and an administrator device of an administrator; and
for each user, obtaining the user account setup data by way of the administrator portal before the server operation of recording.

22. The system of claim 21, wherein the server operations performed by the server processor further comprise
providing a report publication interface by way of the administrator portal;
generating, for each campus-level group by way of the report publication interface, a service report including
the group identifier;
a campus service completion date;
identification of the types of water control points within all rooms associated with the campus-level group; and
for each of the types of water control points within rooms associated with the campus-level group, a tally of the water control points for which service has been completed as of the service completion date; and
including, on one or more of the control point status display pages, a user-selectable report icon; and
responsive to a supervisory user selecting the report icon, providing access to the service report only for campus-level groups associated with rooms corresponding to the location permissions of the icon-selecting supervisory user.

23. The system of claim 22, wherein
the service input data includes room status, the room status being selected from the group consisting of unserviceable, comeback and serviced; and
the admin portal is configured to enable the administrator to review and modify the room status.
